(19) United States
(12) Patent Application Publication
(10) Pub. No.: US 2020/0096591 A1
(43) Pub. Date: Mar. 26, 2020

(54) MAGNETIC RESONANCE IMAGE ACQUISITION METHOD AND MAGNETIC RESONANCE IMAGE DEVICE THEREFOR

(71) Applicant: SAMSUNG ELECTRONICS CO., LTD., Suwon-si (KR)

(72) Inventors: Joon-sung CHOI, Anyang-si (KR); Myung-sung SONG, Hwaseong-si (KR); Dae-ho LEE, Seongnam-si (KR)

(73) Assignee: SAMSUNG ELECTRONICS CO., LTD., Suwon-si (KR)

(21) Appl. No.: 16/472,069

(22) PCT Filed: Oct. 13, 2017
(86) PCT No.: PCT/KR2017/011318

§ 371 Date: Jun. 20, 2019

(30) Foreign Application Priority Data
Jan. 26, 2017 (KR) 10-2017-0012971

(51) Int. Cl. G01R 33/563; G01R 33/48; G01R 33/56; A61B 5/055

(52) U.S. Cl. CPC G01R 33/5635; A61B 5/055; G01R 33/5608; G01R 33/4818

(57) ABSTRACT

Provided is a method of acquiring magnetic resonance (MR) image with respect to an object including a blood vessel by using a three-dimensional (3D) gradient echo sequence, the method including: acquiring k space data with respect to the object based on the 3D gradient echo sequence; and acquiring the MR image with respect to the object based on the acquired k space data, wherein the acquiring of the k space data includes acquiring the k space data based on the 3D gradient echo sequence having a TR (repetition time) that varies according to a value of at least one of a first axis or a second axis of the k space of the k space data.

### TECHNICAL FIELD

The disclosure relates to a magnetic resonance imaging (MRI) method and an MRI apparatus.

More particularly, the disclosure relates to a method of acquiring a magnetic resonance image of an object including a blood vessel and an MRI apparatus.

### BACKGROUND ART

A magnetic resonance imaging (MRI) apparatus images an object by using a magnetic field. Because the MRI apparatus is capable of creating three-dimensional images of bones, discs, joints, ligaments, or the like at a user-desired angle, the MRI apparatus is widely used to make a correct disease diagnosis.

The MRI apparatus acquires a magnetic resonance (MR) signal, reconstructs the acquired MR signal into an image, and outputs the image. In more detail, the MRI apparatus acquires the MR signal by using a high-frequency multi-coil including radio frequency (RF) coils, permanent-magnets, superconducting magnets, gradient coils, etc.

Specifically, a high frequency signal generated by applying a pulse sequence for generating a radio frequency signal to a high frequency multi coil is applied to an object, and a MR image is reconstructed by sampling a magnetic resonance signal generated in response to the applied high-frequency signal.

On the other hand, methods, performed by the MRI apparatus, of imaging a blood vessel include a method of imaging the blood vessel after injecting a contrast agent and a method of imaging the blood vessel without the contrast agent. The method of imaging the blood vessel without the contrast agent includes a time-of-flight (TOF) method of acquiring an MRI using the fact that a newly introduced blood stream generates a signal larger than a tissue in a fixed state. However, in the case of acquiring an image using such a method, a sequence for acquiring the image having a predetermined repetition time (TR) must be repeated to acquire a signal by exciting atoms included in the newly introduced blood stream. Therefore, it takes a comparatively long time to acquire the image, which makes it difficult to speed up an MRI imaging time.

### DESCRIPTION OF EMBODIMENTS

### Technical Problem

Provided are a magnetic resonance imaging (MRI) apparatus and method capable of reducing an acquisition time of an MRI with respect to an object including a blood vessel.

### Solution to Problem

According to an aspect of the disclosure, an apparatus for acquiring magnetic resonance (MR) image with respect to an object comprising a blood vessel by using a 3D gradient echo sequence may include a memory storing the 3D gradient echo sequence; and an image processing unit, wherein the image processing unit is configured to acquire k space data with respect to the object based on the 3D gradient echo sequence and acquire the MR image with respect to the object based on the acquired k space data.

The k space data may be acquired based on the 3D gradient echo sequence having a TR (repetition time) that varies according to a value of at least one of a first axis or a second axis of a k space of the k space data.

### Advantageous Effects of Disclosure

The embodiments may provide a magnetic resonance imaging (MRI) apparatus and method capable of reducing an acquisition time of a MRI with respect to an object including a blood vessel based on a 3D gradient echo sequence having a repetition time (TR) that varies.

### BRIEF DESCRIPTION OF DRAWINGS

FIG. 1 is a block diagram illustrating a magnetic resonance imaging (MRI) apparatus, according to an embodiment.

FIG. 2 is a diagram for explaining a TR (repetition time) that varies according to at least one of a first axis or a second axis of a k space, according to an embodiment.

FIG. 3 is a schematic view of a pulse sequence, according to an embodiment.

FIG. 4 is a schematic view of a pulse sequence, according to another embodiment.

FIG. 5 is a diagram for explaining a method of determining a radio frequency (RF) pulse flip angle, in correspondence to a TR that varies, according to an embodiment.

FIG. 6 is a diagram for explaining a method of acquiring k space data with respect to an object, based on a multi-slab 3D gradient echo sequence, according to an embodiment.

FIG. 7 is a flowchart illustrating a method of acquiring an MRI with respect to an object including a blood vessel, according to an embodiment.

FIG. 8 is a flowchart illustrating a method of acquiring an MRI with respect to an object including a blood vessel, according to another embodiment.

FIG. 9 is a schematic diagram of an MRI system.

### BEST MODE

According to an aspect of the disclosure, an apparatus for acquiring magnetic resonance (MR) image with respect to an object comprising a blood vessel by using a 3D gradient echo sequence may include a memory storing the 3D gradient echo sequence; and an image processing unit, wherein the image processing unit is configured to acquire k space data with respect to the object based on the 3D gradient echo sequence and acquire the MR image with respect to the object based on the acquired k space data.

The k space data may be acquired based on the 3D gradient echo sequence having a TR (repetition time) that varies according to a value of at least one of a first axis or a second axis of a k space of the k space data.

According to another aspect of the disclosure, a method of acquiring magnetic resonance (MR) image with respect to an object comprising a blood vessel by using a three-dimensional (3D) gradient echo sequence may include acquiring k space data with respect to the object based on the 3D gradient echo sequence; and acquiring the MR image with respect to the object based on the acquired k space data, wherein the acquiring of the k space data includes acquiring the k space data based on the 3D gradient echo sequence having a TR (repetition time) that varies according to a value of at least one of a first axis or a second axis of the k space of the k space data.

According to another aspect of the disclosure, a computer-readable recording medium having recorded thereon a program for executing the method on a computer is provided.

### Mode of Disclosure

The present specification describes principles of the disclosure and sets forth embodiments thereof to clarify the scope of the disclosure and to allow those of ordinary skill in the art to implement the embodiments. The present embodiments may have different forms.

Like reference numerals refer to like elements throughout. The present specification does not describe all components in the embodiments, and common knowledge in the art or the same descriptions of the embodiments will be omitted below. The term “part” or “portion” may be implemented using hardware or software, and according to embodiments, one “part” or “portion” may be formed as a single unit or element or include a plurality of units or elements. Hereinafter, the principles and embodiments of the disclosure will be described in detail with reference to the accompanying drawings.

In the present specification, an “image” may include a medical image acquired by a magnetic resonance imaging (MRI) apparatus, a computed tomography (CT) apparatus, an ultrasound imaging apparatus, an X-ray apparatus, or another medical imaging apparatus.

Furthermore, in the present specification, an “object” may be a target to be imaged and include a human, an animal, or a part of a human or animal. For example, the object may include a body part (an organ) or a phantom.

An MRI system acquires an MR signal and reconstructs the acquired MR signal into an image. The MR signal denotes a radio frequency (RF) signal emitted from the object.

In the MRI system, a main magnet creates a static magnetic field to align a magnetic dipole moment of a specific atomic nucleus of the object placed in the static magnetic field along a direction of the static magnetic field. A gradient coil may generate a gradient magnetic field by applying a gradient signal to a static magnetic field and induce resonance frequencies differently according to each region of the object.

An RF coil may emit an RF signal to match a resonance frequency of a region of the object whose image is to be acquired. Furthermore, when gradient magnetic fields are applied, the RF coil may receive MR signals having different resonance frequencies emitted from a plurality of regions of the object. Though this process, the MRI system may acquire an image from an MR signal by using an image reconstruction technique.

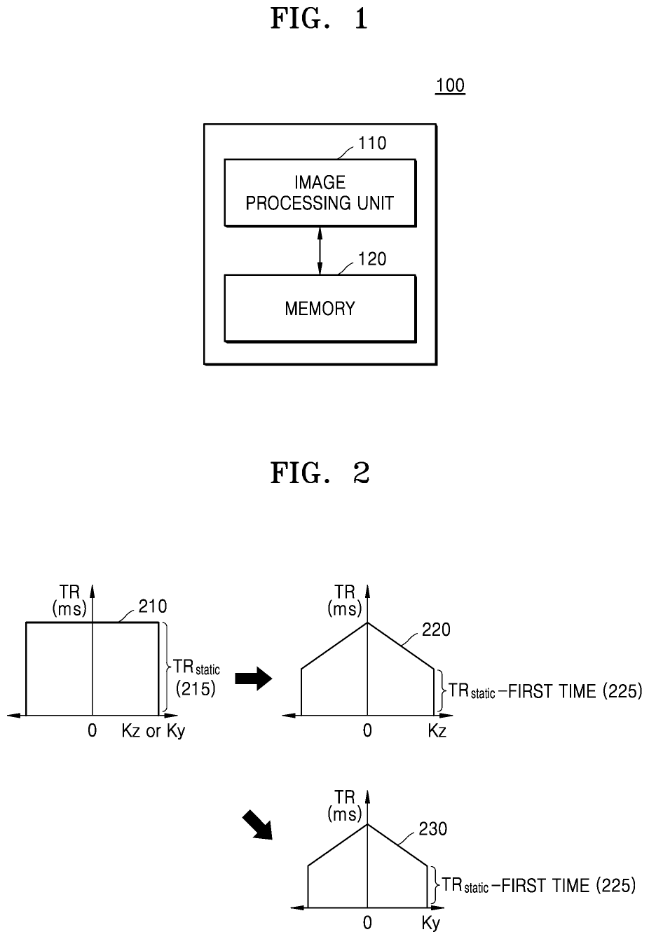

FIG. 1 is a block diagram illustrating a magnetic resonance imaging (MRI) apparatus 100, according to an embodiment.

The MRI apparatus 100 of FIG. 1 may be an apparatus for acquiring an MRI with respect to a blood vessel without using a contrast agent by using a 3D gradient echo sequence.

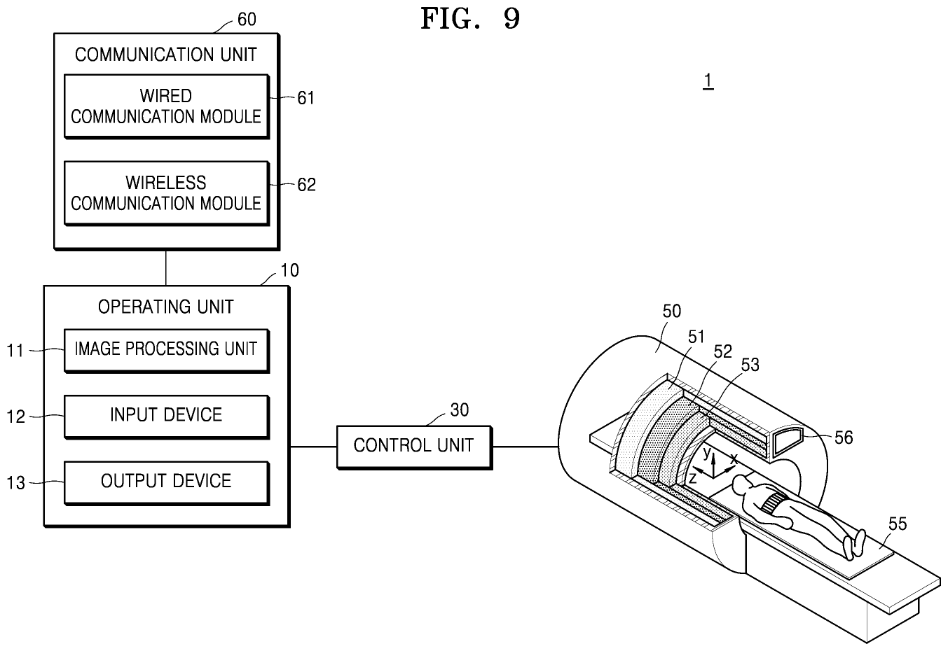

The MRI apparatus 100 according to an embodiment may include an image processing unit 110 and a memory 120. The image processing unit 110 may include at least one processor (not shown). The image processing unit 110 may also correspond to one or a combination of the image processing unit 11 and the control unit 30 shown in FIG. 9, which will be described later.

The image processing unit 110 acquires k space data with respect to an object based on the 3D gradient echo sequence, and acquires an MRI with respect to the object based on the acquired k space data.

Also, the image processing unit 110 acquires the k space data based on the 3D gradient echo sequence having a TR (repetition time) that varies according to at least one of a first axis or a second axis of a k space of the k space data.

In an embodiment, the image processing unit 110 may acquire the k space data with respect to a plurality of slabs included in field of view (FOV) of the object, based on a multi-slab 3D gradient echo sequence, and acquire the MRI with respect to the object based on the acquired k space data.

According to an embodiment, the 3D gradient echo sequence may be a pulse sequence according to a 3D-TOF (time of flight) technique for imaging an MRI of a blood vessel without using the contrast agent.

The 3D-TOF technique may be a technique of imaging a blood vessel image by using a phenomenon in which atoms in tissues of a predetermined volume in field of view (FOV) are saturated by a saturation pulse, and then atoms in blood that is newly introduced into the predetermined volume (that is not influenced by the saturation pulse) are excited by an RF pulse, and thus the atoms in blood emit signals of a greater intensity than those of the atoms in tissue.

In particular, the 3D-TOF technique using the multi-slab divides field of view (FOV) of the object to be imaged into a plurality of volume regions having a constant thickness, images an MRI for each volume region, and then reconstructs the image as one entire volume through a correction process. Such a 3D-TOF technique using the multi-slab has an advantage in that it may acquire a signal of blood having a relatively high contrast. However, because a TR of a sequence is fixed, it is difficult to reduce an imaging time, and because it is based on the characteristic that blood is newly introduced into the plurality of volume regions, it is difficult to use a technique of simultaneously imaging several regions, making it difficult to speed up the 3D-TOF technique using the multi-slab.

According to an embodiment, the image processing unit 110 may acquire k space data with respect to a plurality of slabs included in field of view (FOV) of the object, based on a multi-slab 3D gradient echo sequence having the TR that varies according to at least one of the first axis or the second axis of the k space of the k space data with respect to each of the plurality of slabs. According to an embodiment, the image processing unit 110 may shorten an acquisition time of the MRI with respect to the object including a blood vessel by applying the TR that varies when acquiring the k space data with respect to each of the plurality of slabs.

For example, as magnitude of a value of at least one of the first axis or the second axis of the k space of the k space data with respect to each of the plurality of slabs included in field of view (FOV) of the object increases, the image processing unit 110 may acquire the k space data with respect to with respect to each of the plurality of slabs based on the multi-slab 3D gradient echo sequence having the decreasing TR.

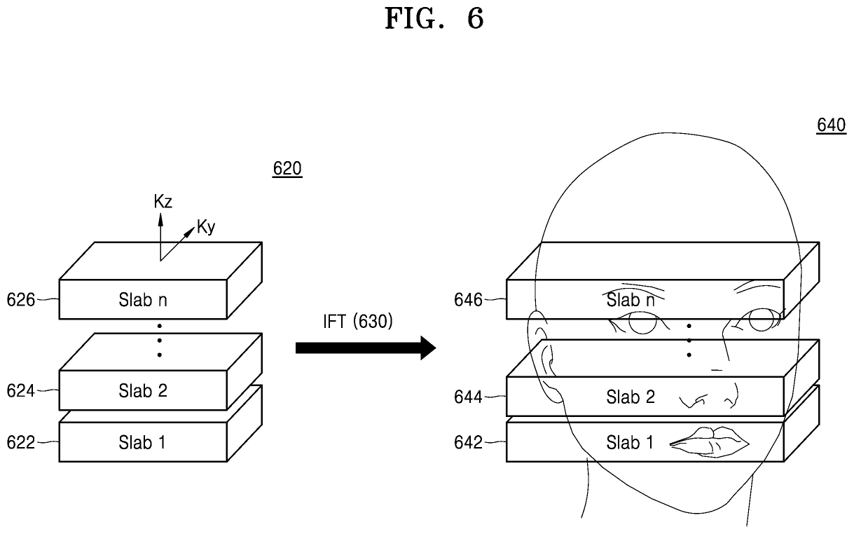

Accordingly, the image processing unit 110 may reduce the entire image acquisition time as compared with the case of using a sequence having a fixed TR when imaging an image of an object including a blood vessel. In this regard, a more detailed description will be provided below with reference to FIG. 6.

Also, a description and an embodiment of the ‘3D gradient echo sequence’ herein may be applied to a ‘multi-slab 3D gradient echo sequence’, and a description and an example of the ‘multi-slab 3D gradient echo sequence’, and a description and an embodiment of the ‘ multi-slab 3D gradient echo sequence’ herein may be applied to the ‘3D gradient echo sequence’.

In an embodiment, the image processing unit 110 may acquire the k space data based on the 3D gradient echo sequence having the TR decreasing as the magnitude of the value of at least one of the first axis or the second axis of the k space of the k space data increases.

In the specification, the first axis and the second axis of the k space may correspond to a z axis (a slice encoding axis) and a y axis (a phase encoding axis), respectively, of the k space.

The image processing unit 110 according to an embodiment may acquire the k space data based on a 3D gradient echo sequence having a TR decreasing as a value of at least one of the z axis or the y axis increases from the center of the k-space. In an embodiment, the image processing unit 110 may acquire the k space data based on a 3D gradient echo sequence having a TR gradually decreasing as the value of at least one of the z-axis or the y-axis corresponds to a high frequency. In this regard, a more detailed description will be provided below with reference to FIG. 2.

Also, in an embodiment, the image processing unit 110 may acquire the k space data based on a 3D gradient echo sequence having a TR decreasing by a first time as the magnitude of the value of at least one of the first axis or the second axis of the k space of the k space data increases. Also, the image processing unit 110 may determine the first time based on a dead time (or an empty space) of the 3D gradient echo sequence.

Also, the image processing unit 110 may determine a TR based on a characteristic or a type of the object from which MRI is to be acquired. Hereinafter, the TR determined based on the characteristic or the type of the object is referred to as TR_{static}.

In a general case, there is a problem that an MRI with respect to an object is acquired based on a sequence to which a fixed value TR_{static} is applied, and accordingly, it is difficult to shorten the acquisition time of the MRI. However, in general, TR_{static} may be a time longer than an active time (a data acquisition time) at which a gradient magnetic field necessary for cross-section selection G_{z}, phase encoding G_{y}, and frequency encoding Gₓ is applied to the object, and the acquisition time of the MRI may be reduced within a range of the dead time which means a remaining time minus the active time from TR_{static}.

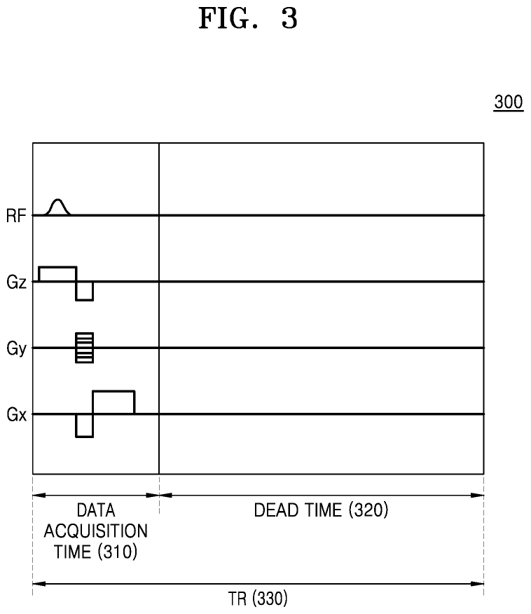

For example, when TR_{static} is 20 ms with respect to the object including blood and the active time is 10 ms, the dead time may be 10 ms. Accordingly, as the magnitude of the value of at least one of the first axis or the second axis of the k space of the k space data increases, the image processing unit 110 according to an embodiment may acquire the k space data based on the 3D gradient echo sequence in which the TR varies from 20 ms to 10 ms reduced by 10 ms equal to the dead time. In this regard, a more detailed description will be provided below with reference to FIG. 3.

In an embodiment, the image processing unit 110 may acquire the k space data with respect to the object based on a 3D gradient echo sequence including a vein spoil block.

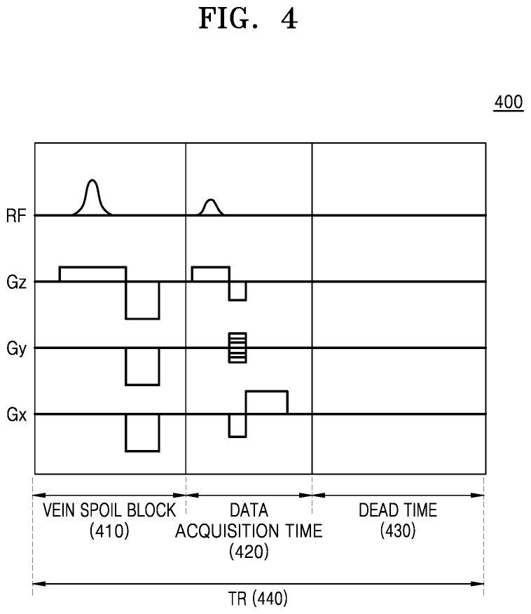

In a magnetic resonance (MR) signal with respect to the object including blood vessel, an image that a user desires to acquire may be the MR signal due to blood flowing in the artery of the object. In this case, the image processing unit 110 may acquire the k space data with respect to the object based on the 3D gradient echo sequence including the vein spoil block for removing the MR signal due to blood flowing in the vein of the object. Because the vein spoil block is added to a time other than the active time (the data acquisition time), when the 3D gradient echo sequence further includes the vein spoil block, the dead time included in the TR may be shorter as compared with the case where the 3D gradient echo sequence includes no vein spoil block. In this regard, a more detailed description will be provided below with reference to FIG. 4.

According to an embodiment, the image processing unit 110 may change other sequence parameters to correspond to the TR that varies so as to minimize quality degradation of the MRI due to the TR that varies.

For example, the image processing unit 110 may change a RF pulse flip angle, TE (echo time), and a dwell time to correspond to the TR that varies.

According to an embodiment, intensity of MR signals of the blood vessel of the object and surrounding tissues of the blood vessel acquired by the image processing unit 110 may be calculated by Equation 1 below.

Sⱼ=M₀sinθ[f_{z,SS}+(cosθ·exp(−TR/T₁))^{j-1}(1−f_{z,SS})]exp(−TE/T₂*) (f_{z,SS}=(1−exp(−TR/T₁))/(1−cosθ·exp(−TR/T₁))) [Equation 1]

In Equation 1 above, j denotes the number of times the RF pulse is received by the blood vessel of the object and surrounding tissues of the blood vessel, M₀ denotes size of a static field, θ denotes the RF pulse flip angle, and f denotes a frequency. In Equation 1 above, because T1 and T2* denotes constant values caused by physical characteristics of a material included in the object, the intensity of the MR signal may depend on the TR, the RF pulse flip angle, and the TE that are sequence parameters.

Accordingly, as the TR varies, the image processing unit 110 according to an embodiment may determine a RF pulse flip angle that may maintain the intensity of the MR signal constant according to Equation 1 above and apply the RF pulse flip angle determined according to the TR that varies to the sequence, thereby acquiring the MR signal of a constant intensity.

Also, according to an embodiment, as the TR varies, the image processing unit 110 may determine a TE that may maintain the intensity of the MR signal constant according to Equation 1 above and apply the RF pulse flip angle determined according to the TR that varies to the sequence, thereby acquiring the MR signal of a constant intensity.

Accordingly, according to the embodiments, while the image acquisition time may be reduced by applying the TR that varies to the sequence, the MR signal of a constant intensity may be acquired by changing and applying values of other image parameters, and quality of an image acquired based on the MR signal may be maintained.

In an embodiment, the image processing unit 110 may determine the RF pulse flip angle that may allow a contrast between a signal by the blood vessel of the object and a signal by the surrounding tissues of the blood vessel to maintain constant, in correspondence to the TR that varies.

For example, as the TR varies, the image processing unit 110 may determine size of the RF pulse flip angle that may allow the contrast between the signal by the blood vessel of the object and the signal by the surrounding tissues of the blood vessel to maintain constant to a predetermined value.

The contrast may be calculated as the intensity of the MR signal of the surrounding tissues of the blood vessel of the object relative to the intensity of the MR signal of the blood vessel. The predetermined value may be a value determined by the image processing unit 110, a value received from an external server, or a value received from the user. For example, the image processing unit 110 may determine the contrast of the signal acquired based on the sequence having TR_{static}, which is determined based on the characteristic or the type of the object that is a target of an image acquisition, as the predetermined value.

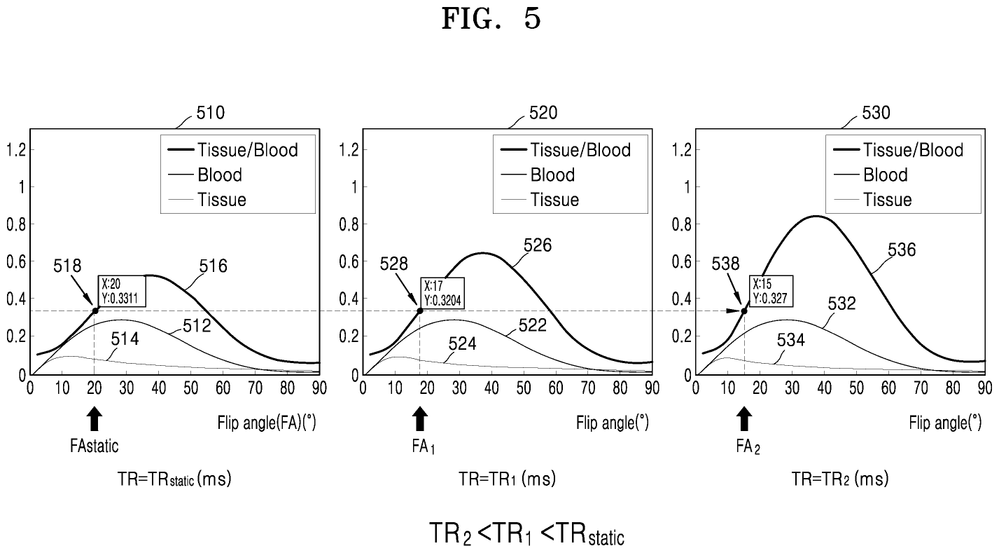

For example, when the MRI of the object including the blood vessel is acquired based on the sequence having TR_{static}, the intensity of the signal of the surrounding tissues relative to the intensity of the signal of the blood vessel may be about 0.3 (30%). Accordingly, the image processing unit 110 may determine the size of the RF pulse flip angle that may allow the contrast between the signal of the surrounding tissues relative to the signal of the blood vessel to be about 0.3 in correspondence to the TR that varies. In this regard, a more detailed description will be provided below with reference to FIG. 5.

Further, in an embodiment, the image processing unit 110 may acquire the k space data with respect to the object by a 3D gradient echo sequence having the TR that varies and the determined RF pulse flip angle, based on the determined RF pulse flip angle.

In an embodiment, the image processing unit 110 may determine at least one of the TE (echo time) or a dwell time that may allow the contrast between the signal by the blood vessel of the object and the signal by the surrounding tissues of the blood vessel to maintain constant, in correspondence to the TR that varies.

Also, in an embodiment, the image processing unit 110 may acquire the k space data with respect to the object by a 3D gradient echo sequence having at least one of the TR that varies, the determined TE, or the determined dwell time, based on at least one of the determined TE or the determined dwell time.

The memory 120 stores the 3D gradient echo sequence.

In an embodiment, the memory 120 may store the multi-slab 3D gradient echo sequence.

Further, in an embodiment, the memory 120 may store various types of sequences and image parameter values for acquiring the MR signal from the object.

In an embodiment, the memory 120 may store various data or programs, input/output MR signals, acquired MRIs, and the like for driving and controlling the MRI apparatus 100.

FIG. 2 is a diagram for explaining a TR (repetition time) TR that varies according to at least one of a first axis or a second axis of a k space according to an embodiment.

Referring to FIG. 2, each of graphs 210 to 230 shows TR values with respect to the z-axis or the y-axis of the k space of k space data.

Graph 210 shows that the TR values with respect to the z-axis or the y-axis of the k-space of the k space data are constant as TR_{static} 215 regardless of a z-axis or y-axis value of the k-space.

Generally, a sequence having a fixed TR (=TR_{static} 215) is used when an MRI of an object including a blood vessel is acquired. Based on a 3D gradient echo sequence, when 3D k space data with respect to the object is acquired, line data with respect to a specific location (Ky, Kz)=(a, b) of the k space may be acquired by applying one RF pulse. Also, the entire k space volume data may be acquired by repeating a RF pulse a plurality of times at an interval of the TR_{static} 215.

The TR_{static} 215 may be determined based on a characteristic or a type of the object from which an MRI is to be acquired. For example, the TR_{static} 215 of the sequence used when the MRI of the object including the blood vessel is acquired may be 20 ms. Accordingly, in a general case, when the MRI of the object including the blood vessel is acquired, the MRI apparatus 100 may acquire a MR signal with respect to the object by applying the RF pulse at an equal interval of 20 ms regardless of an increase in a frequency value from the center of the k space of the k space data to a z-axis direction (a slice encoding direction) or a y-axis direction (a phase encoding direction).

On the other hand, in case where the TR of the sequence is reduced when an MRI is acquired, because nuclei receive the RF pulse again before longitudinal axis magnetization of a nucleus spindle included in the object is completely recovered, intensity of a signal emitted from a nucleus and an image contrast may be reduced. However, a signal-to-noise ratio and an image contrast in most MRIs are affected by a low frequency component of the k-space, and a high frequency component involves a detail of the image.

Accordingly, the MRI apparatus 100 according to the embodiment may acquire the MRI using a sequence having the TR decreasing as a value of at least one of the z-axis or the y-axis increases to a value of a high frequency from the center of the k space of the k space data, so as to maintain identity of the signal-to-noise ratio and the image contrast of the image acquired based on the sequence having the fixed TR (=TR_{static} 215) and the image acquired based on the sequence having the TR that varies.

Graph 220 of FIG. 2 shows a TR decreasing by TR_{static}—a first time 225 in the TR_{static} 215 as a value of the z-axis increases from the center of the k space of the k space data. Graph 230 shows a TR decreasing by TR_{static}—the first time 225 in the TR_{static} 215 as a value of the y-axis increases from the center of the k space of the k space data. An internal area of graphs 210 to 230 shown in FIG. 2 may be proportional to an image acquisition time.

For example, the general TR_{static} 215 of a sequence used when the MRI of the object including the blood vessel is acquired may be 20 ms, and the first time 225 may be determined to be 10 ms based on a dead time of the sequence. In this case, the MRI apparatus 100 according to an embodiment may acquire the MRI based on a 3D gradient echo sequence having the TR deceasing from 20 ms (TR_{static} 215) to 10 ms (TR_{static}—the first time 225) as the value of the z-axis or the y-axis of the k space of the k space data increases (corresponding to a high frequency).

Accordingly, in case of using the sequence having the TR that varies, an image acquisition time may be reduced by 25% as compared with the case where TR is fixed (the internal area of graphs 220 and 230 is reduced by 25% as compared with the internal area of graph 210).

FIG. 3 is a schematic view 300 of a 3D gradient echo sequence, according to an embodiment.

Referring to FIG. 3, a TR 330 of the 3D gradient echo sequence may include an active time corresponding to a data acquisition time 310 and a dead time 320. The dead time 320 may be a time excluding a time for applying a cross-section selective gradient magnetic field G_{z}, the phase encoding gradient magnetic field G_{y}, and a frequency encoding gradient magnetic field Gₓto acquire k space data in the TR 330.

In an embodiment, the MRI apparatus 100 may determine a first time based on the dead time 320 included in the TR 330.

In an embodiment, the MRI apparatus 100 may determine a time corresponding to the dead time 320 as the first time. For example, when the dead time 320 is 10 ms, the MRI apparatus 100 may determine the first time to be 10 ms.

In an embodiment, the first time may be the dead time 320.

In an embodiment, the MRI apparatus 100 may determine a time value of one of values included in a range of more than 0 to less than the dead time 320 (0<the first time<the dead time 320) as the first time. Further, the MRI apparatus 100 may determine, based on a predetermined criterion, a time value of one of the values included in the range as the first time.

For example, when a relatively fine image needs to be acquired, the MRI apparatus 100 may determine a relatively small value among the values included in the range as the first time. Also, when an image in which a relatively detailed expression is not important is to be acquired, the MRI apparatus 100 may determine a relatively large value among the values included in the range as the first time. Also, a predetermined criterion for the MRI apparatus 100 to determine the first time may be stored in the memory 120, received from a user, or received from an external server (not shown).

For example, a TR of a sequence used to acquire an MRI of an object including a blood vessel may be 20 ms, and the dead time 320 of 10 ms may be included in the TR of 20 ms. In this case, the MRI apparatus 100 according to an embodiment may determine 10 ms corresponding to the dead time 320 as the first time. Also, the MRI apparatus 100 according to another embodiment may determine a time value of one of values included in a range of more than 0 to less than 10 ms as the first time according to a predetermined criterion.

FIG. 4 is a schematic view 400 of a 3D gradient echo sequence, according to another embodiment.

FIG. 4 shows the 3D gradient echo sequence further including a vein spoil block 410, as compared to FIG. 3. In an embodiment, a TR 440 of the 3D gradient echo sequence may include a time by the vein spoil block 410, a data acquisition time 420, and a dead time 430.

In an embodiment, the dead time 430 in case where the 3D gradient echo sequence further includes the vein spoil block 410 may correspond to a remaining time excluding the data acquisition time 420 and the time by the vein spoil block in the TR 440.

In general, the vein spoil block 410 may be added using the dead time 430 in the TR 440. Accordingly, the dead time 430 in case where the 3D gradient echo sequence further includes the vein spoil block 410 may include a time reduced by the time by the vein spoil block 410 from the dead time 320 (of FIG. 3) in case where the 3D gradient echo sequence does not include the vein spoil block 410.

In case where the 3D gradient echo sequence further includes the vein spoil block 410, a configuration for determining the first time based on the dead time 430 may correspond to a configuration for determining the first time based on the dead time 320. Therefore, a description redundant with the description in FIG. 3 is omitted.

FIG. 5 is a diagram for explaining a method of determining an RF pulse flip angle, in correspondence to a TR that varies, according to an embodiment.

Referring to FIG. 5, graphs 510 through 530 respectively illustrate signals 512, 522, and 523 of a blood vessel of an object with respect to the RF pulse flip angle, signals 514, 524, and 534 by surrounding tissues of the blood vessel, and contrasts 516, 526, and 536 of the signals 514, 524, and 534 by the surrounding tissues relative to the signals 512, 522, and 523 of the blood vessel according to an embodiment when TR=TR_{static}, TR=TR1, and TR=TR2.

In general, a TR of a 3D gradient sequence used to acquire an MRI of the object including the blood vessel is denoted by TR_{static}, and the RF pulse flip angle FA is denoted by FA_{static.} For example, the sequence used to acquire the MRI of the object including the blood vessel may be that TR_{static}=20 ms and FA_{static}=20°.

In an embodiment, the MRI apparatus 100 may determine a contrast (hereinafter referred to as a ‘reference contrast’) of a signal by surrounding tissues relative to a signal of a blood vessel when the TR of the 3D gradient sequence is TR_{static} and the FA is FA_{static}. Referring to graph 510, a reference contrast value 518 may be about 0.3 when TR is TR_{static} and FA is FA_{static}.

The MRI apparatus 100 according to an embodiment may determine the FA that may allow the contrast of the signal by the blood vessel of the object and the signal by the surrounding tissues of the blood vessel of the object to have a value corresponding to the determined reference contrast in correspondence to the TR that varies.

Also, when there are a plurality of FA values that may allow the contrast of the signal by the blood vessel of the object and the signal by the surrounding tissues of the blood vessel of the object to have the value corresponding to the determined reference contrast, the MRI apparatus 100 according to an embodiment may determine a FA corresponding to the smallest value among the plurality of FA values as the FA corresponding to the TR that varies.

Graph 520 shows the signal 522 of the blood vessel of the object, the signal 524 of the surrounding tissues of the blood vessel, and the contrast 526 the signal 524 by the surrounding tissues relative to the signal 522 of the blood vessel with respect to the RF pulse flip angle when the TR decreases from TR_{static} to TR₁(ms). Referring to graph 520, FA₁ at a point 528, which has the same contrast value as the reference contrast value 518, may correspond to 17°. Accordingly, the MRI apparatus 100 may determine the FA to 17° when the TR varies to TR₁(ms).

Also, graph 530 shows the signal 532 of the blood vessel of the object, the signal 534 of the surrounding tissues of the blood vessel, and the contrast 536 the signal 534 by the surrounding tissues relative to the signal 532 of the blood vessel with respect to the RF pulse flip angle when the TR decreases from TR₁(ms) to TR₂(ms). Referring to graph 530, FA₂ at a point 538, which has the same contrast value as the reference contrast value 518, may correspond to 15°. Accordingly, the MRI apparatus 100 may determine the FA to 15° when the TR varies to TR₂(ms).

In FIG. 5, although the MRI apparatus 100 determines the reference contrast based on TR_{static} and FA_{static}, and determines the RF pulse flip angle FA in correspondence to the TR that varies based on the determined reference contrast, the reference contrast may be a value previously stored in the memory 120 of the MRI apparatus 100 according to the type of the object.

According to the embodiments, the MRI apparatus 100 applies the FA to a sequence that may allow the contrast of the signal by the blood vessel of the object and the signal by the surrounding tissues of the blood vessel of the object to maintain constant along with the TR that varies, thereby acquiring the MRI having relatively equal quality while shortening a time for acquiring the MRI by applying the TR that varies.

FIG. 6 is a diagram for explaining a method of acquiring k space data 620 with respect to an object, based on a multi-slab 3D gradient echo sequence, according to an embodiment.

Referring to FIG. 6, the MRI apparatus 100 may acquire the k space data 620 divided into using a plurality of slabs Slab 1 622, Slab 2 624, . . . , and Slab n 626 with respect to the object based on the multi-slab 3D gradient echo sequence.

In an embodiment, the MRI apparatus 100 may acquire the k space data 620 with respect to the plurality of slabs Slab 1 622, Slab 2 624, . . . , and Slab n 626 based on the multi-slab 3D gradient echo sequence having a TR that varies according to a value of at least one of a z-axis K_{z} or a y-axis K_{y} of a k space with respect to each of a plurality of slabs 622, 624, . . . , 626.

That is, the MRI apparatus 100 may acquire the k space data 620 with respect to the slab Slab 1 622 based on a sequence having the TR that varies according to the value of at least one of the z-axis K_{z} or the y-axis K_{y} of the k space with respect to the k space data 620 with respect to the Slab 1 622 when acquiring the k space data 620 with respect to the slab Slab 1 622. Also, the MRI apparatus 100 may acquire the k space data 620 with respect to the slab Slab 2 624 based on the sequence having the TR that varies according to the value of at least one of the z-axis K_{z} or the y-axis K_{y} of the k space with respect to the k space data 620 with respect to the Slab 2 624 when acquiring the k space data 620 with respect to the slab Slab 2 624. Likewise, the MRI apparatus 100 may acquire the k space data 620 with respect to the slab Slab n 626 based on the sequence having the TR that varies according to the value of at least one of the z-axis K_{z} or the y-axis K_{y} of the k space with respect to the k space data 620 with respect to the Slab n 626 when acquiring the k space data 620 with respect to the slab Slab n 626.

The MRI apparatus 100 may perform inverse Fourier transform 630 on the k space data 620 acquired by being divided into the plurality of slabs 622, 624, . . . , 626 to acquire volume data with respect to the plurality of slabs 622, 624, . . . , 626 included in an image acquisition region 640 of the object in a time domain.

According to the embodiments, the MRI apparatus 100 may acquire a blood vessel image having a relatively high image contrast based on the multi-slab 3D gradient echo sequence, while shortening the image acquisition region 640 by applying the TR that varies when acquiring the k space data 620 for each of the slabs 622, 624, . . . , 626.

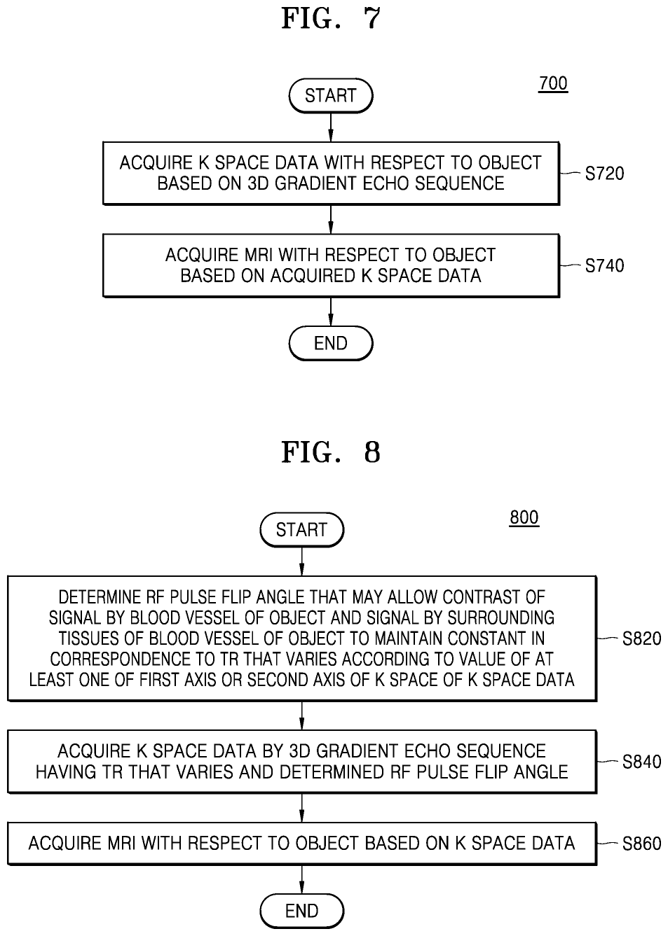

FIG. 7 is a flowchart illustrating a method 700 of acquiring an MRI with respect to an object including a blood vessel, according to an embodiment.

The method 700 of acquiring the MRI with respect to the object including the blood vessel according to an embodiment shown in FIG. 7 may be performed through the MRI apparatus 100 according to the embodiment described above.

The MRI apparatus 100 acquires k space data with respect to the object including the blood vessel based on a 3D gradient echo sequence (S720).

The MRI apparatus 100 acquires the MRI with respect to the object based on the acquired k space data (S740).

Operation S720 includes an operation, performed by the MRI apparatus 100, of acquiring the k space data based on the 3D gradient echo sequence having a TR that varies according to at least one of a first axis or a second axis of a k space of the k space data.

FIG. 8 is a flowchart illustrating a method 800 of acquiring an MRI with respect to an object including a blood vessel, according to another embodiment.

The method 800 of acquiring the MRI with respect to the object including the blood vessel according to another embodiment shown in FIG. 8 may be performed through the MRI apparatus 100 according to the embodiment described above.

Also, operations S820 and S840 of the method 800 of acquiring the MRI with respect to the object including the blood vessel according to another embodiment shown in FIG. 8 may be operations included in operation S720 shown in FIG. 7, and operation S860 may correspond to operation S740 shown in FIG. 7.

The MRI apparatus 100 may determine the RF pulse flip angle FA that may allow a contrast of a signal by the blood vessel of the object and a signal by surrounding tissues of the blood vessel of the object to maintain constant in correspondence to a TR that varies according to a value of at least one of a first axis or a second axis of a k space of k space data (S820).

The MRI apparatus 100 may acquire the k space data with respect to the object based on a 3D gradient echo sequence having the TR that varies according to the value of at least one of the first axis or the second axis of the k space of the k space data and the determined FA (S840).

The MRI apparatus 100 may acquire the MRI with respect to the object based on the acquired k space data (S860).

FIG. 9 is a schematic diagram of an MRI system 1. Referring to FIG. 9, the MRI system 1 may include an operating unit 10, a controller 30, and a scanner 50. The controller 30 may be independently implemented as shown in FIG. 9. Alternatively, the controller 30 may be separated into a plurality of sub-components and incorporated into the operating unit 10 and the scanner 50 in the MRI system 1. Operations of the components in the MRI system 1 will now be described in detail.

The scanner 50 may be formed to have a cylindrical shape (e.g., a shape of a bore) having an empty inner space into which an object may be inserted. A static magnetic field and a gradient magnetic field are created in the inner space of the scanner 50, and an RF signal is emitted toward the inner space.

The scanner 50 may include a static magnetic field generator 51, a gradient magnetic field generator 52, an RF coil unit 53, a table 55, and a display 56. The static magnetic field generator 51 creates a static magnetic field for aligning magnetic dipole moments of atomic nuclei of the object in a direction of the static magnetic field. The static magnetic field generator 51 may be formed as a permanent magnet or superconducting magnet using a cooling coil.

The gradient magnetic field generator 52 is connected to the controller 30 and generates a gradient magnetic field by applying a gradient to a static magnetic field in response to a control signal received from the controller 30. The gradient magnetic field generator 52 includes X, Y, and Z coils for generating gradient magnetic fields in X-, Y-, and Z-axis directions crossing each other at right angles and generates a gradient signal according to a position of a region being imaged so as to differently induce resonance frequencies according to regions of the object.

The RF coil unit 53 connected to the controller 30 may emit an RF signal toward the object in response to a control signal received from the controller 30 and receive an MR signal emitted from the object. In detail, the RF coil unit 53 may transmit, toward atomic nuclei of the object having precessional motion, an RF signal having the same frequency as that of the precessional motion, stop transmitting the RF signal, and then receive an MR signal emitted from the object.

The RF coil unit 53 may be formed as a transmitting RF coil for generating an electromagnetic wave having an RF corresponding to the type of an atomic nucleus, a receiving RF coil for receiving an electromagnetic wave emitted from an atomic nucleus, or one transmitting/receiving RF coil serving both functions of the transmitting RF coil and receiving RF coil. Furthermore, in addition to the RF coil unit 53, a separate coil may be attached to the object. Examples of the separate coil may include a head coil, a spine coil, a torso coil, and a knee coil according to a region being imaged or to which the separate coil is attached.

The display 56 may be disposed outside and/or inside the scanner 50. The display 56 is also controlled by the controller 30 to provide a user or the object with information related to medical imaging.

Furthermore, the scanner 50 may include an object monitoring information acquisition unit configured to acquire and transmit monitoring information about a state of the object. For example, the object monitoring information acquisition unit (not shown) may acquire monitoring information related to the object from a camera (not shown) for imaging images of a movement or position of the object, a respiration measurer (not shown) for measuring the respiration of the object, an ECG measurer for measuring the electrical activity of the heart, or a temperature measurer for measuring a temperature of the object and transmit the acquired monitoring information to the controller 30. The controller 30 may in turn control an operation of the scanner 50 based on the monitoring information. Operations of the controller 30 will now be described in more detail.

The controller 150 may control overall operations of the X-ray apparatus 50.

The controller 30 may control a sequence of signals formed in the scanner 50. The controller 30 may control the gradient magnetic field generator 52 and the RF coil unit 53 according to a pulse sequence received from the operating unit 10 or a designed pulse sequence.

A pulse sequence may include all pieces of information required to control the gradient magnetic field generator 52 and the RF coil unit 53. For example, the pulse sequence may include information about a strength, a duration, and application timing of a pulse signal applied to the gradient magnetic field generator

The controller 30 may control a waveform generator (not shown) for generating a gradient wave, i.e., an electrical pulse according to a pulse sequence and a gradient amplifier (not shown) for amplifying the generated electrical pulse and transmitting the same to the gradient magnetic field generator 52. Thus, the controller 30 may control formation of a gradient magnetic field by the gradient magnetic field generator 52.

Furthermore, the controller 30 may control an operation of the RF coil unit 53. For example, the controller 30 may supply an RF pulse having a resonance frequency to the RF coil unit 30 that emits an RF signal toward the object, and receive an MR signal received by the RF control unit 53. In this case, the controller 30 may adjust emission of an RF signal and reception of an MR signal according to an operating mode by controlling an operation of a switch (e.g., a T/R switch) for adjusting transmitting and receiving directions of the RF signal and the MR signal based on a control signal.

The controller 30 may control a movement of the table 55 where the object is placed. Before imaging is performed, the controller 30 may previously move the table 55 in accordance with an imaging part of the object.

The controller 30 may also control the display 56. For example, the controller 30 control the on/off state of the display 56 or a screen to be output on the display 56 according to a control signal.

The controller 30 may be formed as an algorithm for controlling operations of the components in the MRI system 1, a memory (not shown) for storing data in the form of a program, and a processor for performing the above-described operations by using the data stored in the memory. In this case, the memory and the processor may be implemented as separate chips. Alternatively, the memory and processor may be incorporated into a single chip.

The operating unit 10 may control overall operations of the MRI system 1 and include an image processing unit 11, an input device 12, and an output device 13.

The image processing unit 11 may control the memory to store an MR signal received from the controller 30, and generate image data with respect to the object from the stored MR signal by applying an image reconstruction technique by using an image processor.

For example, when a k space (for example, also referred to as a Fourier space or a frequency space) of the memory is filled with digital data to complete k space data, the image processing unit 11 may reconstruct image data from the k space data by applying various image reconstruction techniques (e.g., by performing inverse Fourier transform on the k space data) by using the image processor.

Furthermore, the image processing unit 11 may perform various signal processing operations on MR signals in parallel. For example, the image processor 62 may perform a signal process on a plurality of MR signals received by a multi-channel RF coil in parallel so as to rearrange the plurality of MR signals into image data. Also, the image processing unit 11 may store not only the image data in the memory, or the controller 30 may store the same in an external server via a communication unit 60 as will be described below.

The input device 12 may receive, from the user, a control command for controlling the overall operations of the MRI system 1. For example, the input device 12 may receive, from the user, object information, parameter information, a scan condition, and information about a pulse sequence. The input device 12 may be a keyboard, a mouse, a track ball, a voice recognizer, a gesture recognizer, a touch screen, or any other input device.

The output device 13 may output image data generated by the image processing unit 11. The output device 13 may also output a user interface (UI) configured so that the user may input a control command related to the MRI system 1. The output device 13 may be formed as a speaker, a printer, a display, or any other output device.

Furthermore, although FIG. 9 shows that the operating unit 10 and the controller 30 are separate components, the operating unit 10 and the controller 30 may be included in a single device as described above. Furthermore, processes respectively performed by the operating unit 10 and the controller 30 may be performed by another component. For example, the image processing unit 11 may convert an MR signal received from the controller 30 into a digital signal, or the controller 30 may directly perform the conversion of the MR signal into the digital signal.

The MRI system 1 may further include a communication unit 60 and be connected to an external device (not shown) such as a server, a medical apparatus, and a portable device (e.g., a smartphone, a tablet PC, a wearable device, etc.) via the communication unit 60.

The communication unit 60 may include at least one component that enables communication with an external device. For example, the communication unit 60 may include at least one of a local area communication module (not shown), a wired communication module 61, and a wireless communication module 62.

The communication unit 60 may receive a control signal and data from an external device and transmit the received control signal to the controller 30 so that the controller 30 may control the MRI system 1 according to the received signal.

Alternatively, by transmitting a control signal to an external device via the communication unit 60, the controller 30 may control the external device according to the control signal.

For example, the external device may process data of the external device according to a control signal received from the controller 30 via the communication unit 60.

A program for controlling the MRI system 1 may be installed on the external device and may include instructions for performing some or all of the operations of the controller 30.

The program may be preinstalled on the external device, or a user of the external device may download the program from a server providing an application for installation. The server providing an application may include a recording medium having the program recorded thereon.

The above-described embodiments of the disclosure may be embodied in form of a computer-readable recording medium for storing computer executable command languages and data. The command languages may be stored in form of program codes and, when executed by a processor, may perform a certain operation by generating a certain program module. Also, when executed by a processor, the command languages may perform certain operations of the disclosed embodiments.

While embodiments of the disclosure have been particularly shown and described with reference to the accompanying drawings, it will be understood by those of ordinary skill in the art that various changes in form and details may be made therein without departing from the spirit and scope of the inventive concept as defined by the appended claims. The disclosed embodiments should be considered in descriptive sense only and not for purposes of limitation.

1. An apparatus for acquiring magnetic resonance (MR) image with respect to an object comprising a blood vessel by using a 3D gradient echo sequence, the apparatus comprising:
a memory storing the 3D gradient echo sequence; and
an image processing unit,
wherein the image processing unit is configured to acquire k space data with respect to the object based on the 3D gradient echo sequence and acquire the MR image with respect to the object based on the acquired k space data, and
wherein the k space data is acquired based on the 3D gradient echo sequence having a TR (repetition time) that varies according to a value of at least one of a first axis or a second axis of a k space of the k space data.

2. The apparatus of claim 1,
wherein the image processing unit is further configured to acquire the k space data with respect to a plurality of slabs included in a field of view (FOV) of the object based on a multi-slab 3D gradient echo sequence, and
wherein the k space data with respect to the plurality of slabs is acquired based on the multi-slab 3D gradient echo sequence having the TR that varies according to the value of at least one of the first axis or the second axis of the k space of the k space data with respect to each of the plurality of slabs.

3. The apparatus of claim 1, wherein the TR decreases as a magnitude of the value of at least one of the first axis or the second axis of the k space of the k space data increases.

4. The apparatus of claim 3, wherein the TR decreases by a first time as the magnitude of the value of at least one of the first axis or the second axis of the k space of the k space data increases, and
wherein the first time is determined based on a dead time of the 3D gradient echo sequence.

5. The apparatus of claim 1, wherein the image processing unit is further configured to determine a radio frequency (RF) pulse flip angle that may allow a contrast between a signal caused by the blood vessel of the object and a signal by surrounding tissues of the blood vessel to remain constant, in correspondence to the TR that varies, and
wherein the k space data is acquired based on the 3D gradient echo sequence having the TR that varies and the determined RF pulse flip angle.

6. The apparatus of claim 1, wherein the image processing unit is further configured to determine at least one of a TE (echo time) or a dwell time that allows a contrast between a signal caused by the blood vessel of the object and a signal by surrounding tissues of the blood vessel to remain constant, in correspondence to the TR that varies, and
wherein the k space data is acquired based on the 3D gradient echo sequence having the TR that varies and at least one of the determined TE or dwell time.

7. The apparatus of claim 1, wherein the 3D gradient echo sequence comprises a vein spoil block for removing a signal caused by a vein included in the field of view (FOV) of the object.

8. A method of acquiring magnetic resonance (MR) image with respect to an object comprising a blood vessel by using a three-dimensional (3D) gradient echo sequence, the method comprising:
acquiring k space data with respect to the object based on the 3D gradient echo sequence; and
acquiring the MR image with respect to the object based on the acquired k space data,
wherein the acquiring of the k space data comprises acquiring the k space data based on the 3D gradient echo sequence having a TR (repetition time) that varies according to a value of at least one of a first axis or a second axis of the k space of the k space data.

9. The method of claim 1,
wherein the acquiring of the k space data comprises acquiring the k space data with respect to a plurality of slabs included in a field of view (FOV) of the object based on a multi-slab 3D gradient echo sequence, and
wherein the acquiring of the k space data with respect to the plurality of slabs comprises acquiring the k space data with respect to the plurality of slabs based on the multi-slab 3D gradient echo sequence having the TR that varies according to the value of at least one of the first axis or the second axis of the k space of the k space data with respect to each of the plurality of slabs.

10. The method of claim 8, wherein the TR decreases as a magnitude of the value of at least one of the first axis or the second axis of the k space of the k space data increases.

11. The method of claim 10, wherein the TR decreases by a first time as the magnitude of the value of at least one of the first axis or the second axis of the k space of the k space data increases, and
wherein the first time is determined based on a dead time of the 3D gradient echo sequence.

12. The method of claim 8, wherein the acquiring of the k space data comprises:
determining a radio frequency (RF) pulse flip angle that allows a contrast between a signal caused by the blood vessel of the object and a signal by surrounding tissues of the blood vessel to remain constant, in correspondence to the TR that varies, and
acquiring the k space data based on the 3D gradient echo sequence having the TR that varies and the determined RF pulse flip angle.

13. The method of claim 8, wherein the acquiring of the k space data comprises:
determining at least one of a TE (echo time) or a dwell time that allows a contrast between a signal caused by the blood vessel of the object and a signal by surrounding tissues of the blood vessel to remain constant, in correspondence to the TR that varies, and
acquiring the k space data based on the 3D gradient echo sequence having the TR that varies and at least one of the determined TE or dwell time.

14. The method of claim 8, wherein the 3D gradient echo sequence comprises a vein spoil block for removing a signal caused by a vein included in the field of view (FOV) of the object.

15. A non-transitory computer-readable recording medium having recorded thereon a program for performing the method of claim 8 on a computer.
